(19) United States
(12) Patent Application Publication
(10) Pub. No.: US 2025/0353880 A1
(43) Pub. Date: Nov. 20, 2025

(54) IMPROVED PROCESS FOR THE PREPARATION OF VOCLOSPORIN

(71) Applicant: DR. REDDY’S LABORATORIES LIMITED, Hyderabad (IN)

(72) Inventors: Tania KATSINA, Cambridgeshire (GB); Paola DE LUISI, Cambridgeshire (GB); Graham MEEK, Cambridgeshire (GB); Fabrizio PALUMBO, Cambridgeshire (GB)

(21) Appl. No.: 19/108,346

(22) PCT Filed: Sep. 8, 2023
(86) PCT No.: PCT/IN2023/050851

§ 371 Date: Mar. 3, 2025

(30) Foreign Application Priority Data
Sep. 9, 2022 (IN) 202241051571

(51) Int. Cl. C07K 7/64

(52) U.S. Cl. CPC C07K 7/645

(57) ABSTRACT

The present invention provides an improved process for the preparation of Voclosporin, comprising oxidation of cyclosporin A acetate with potassium osmate and N-methyl morpholine N-oxide (NMIMO) followed by sodium periodate and further conversion to Voclosporin. The present invention also covers crystallization of crude Voclosporin from acetone and heptane mixture.

### CROSS REFERENCE

This application claims priority of Indian patent application IN 202241051571 filed on 9^{th} Sep. 2022.

### FIELD OF INVENTION

The present application relates to an improved process for the preparation of Voclosporin.

### BACKGROUND OF INVENTION

The drug compound having International Non-proprietary Name, “Voclosporin”, has a chemical name of, cyclo{{(6E)-(2S,3R,4R)-3-hydroxy-4-methyl-2-(methylamino)-6,8 -nonadienoyl}-L-2-aminobutyryl-N-methyl-glycyl-N-methyl-L-leucyl-L-valyl-N-methyl-L-leucyl-L-alanyl-D-alanyl-N-methyl-L-leucyl-N-methyl-L-leucyl-N-methyl-L-valyl} and is represented by the structure of formula (I) as shown below.

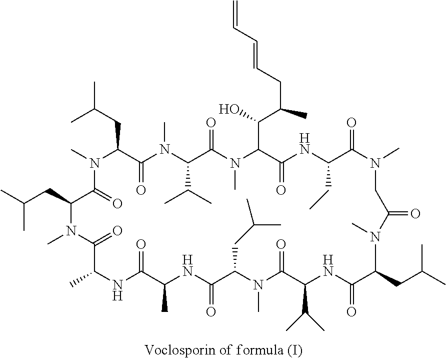 [SMILES: C=C/C=C/C[C@@H](C)[C@@H](O)C1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

Voclosporin is a calcineurin-inhibitor immunosuppressant drug and indicated in combination with a background immunosuppressive therapy regimen for the treatment of adult patients with active lupus nephritis.

U.S. Pat. No. 7,521,421 B2 (Assigned to Isotechnica) discloses a process for the preparation of Voclosporin which comprises reaction of cyclosporin A with acetic anhydride and then oxidizing the obtained acetyl cyclosporin A of formula (III) with sodium periodate and then osmium tetroxide in mixture of dioxane and water. Further, the obtained aldehyde compound of formula (IV) is reacted with corresponding phosphonium ylide followed by removal of acetyl group using potassium carbonate to get Voclosporin.

The main drawback of the above prior art process is direct handling of osmium tetroxide which is toxic. The use of osmium tetroxide does not make the process economically viable as osmium tetroxide is an expensive reagent and moreover the reported yield is only 79%.

U.S. Pat. No. 7,332,472 B2 (Assigned to Isotechnica) discloses synthesis of Voclosporin, comprising oxidation of compound of formula (III) using sodium periodate (NaIO₄) in presence of Ruthenium trichloride (RuCl₃) to afford acetyl protected cyclosporin A aldehyde compound of formula (IV) which is further converted to Voclosporin. This patent also involves the use of traditional chromatography and solvent distillation techniques for isolation of Voclosporin.

The drawback of the above prior art process is lower yield (68%) of aldehyde intermediate compound of formula (IV) and use of chromatographic methods for purification and isolation of Voclosporin which is tedious, expensive and accordingly not viable at industrial scale.

U.S. Pat. No. 7,799,756 B2 (Assigned to Albany Molecular Research) discloses preparation of Voclosporin, comprising oxidation of acetyl protected cyclosporin A compound of formula (III) using Ozone gas. Further, this patent involves the use of semi preparative HPLC for purification/isolation of Voclosporin.

The main drawback in the above prior art process is use of Ozone gas which is toxic and requires dedicated facility to handle. Moreover, ozonolysis needs to be performed under cryogenic conditions making the process tedious and not viable at industrial scale.

Therefore, there remains a need to develop a process which is cost-effective, does not use toxic reagents and industrial viable.

### Advantages of the Present Invention

The reaction mixture containing potassium osmate and aqueous N-methyl morpholine N-oxide solution (NMMO) in-situ generates osmium tetroxide (OsO4) which enables oxidation of compound of formula (III) and avoids direct contact with toxic osmium tetroxide. This makes the present process safe and cost-effective.
Use of potassium osmate and aqueous NMMO increases the yield of the resulting aldehyde compound to almost quantitative yield. Also, the present process avoids column chromatography of the intermediate and hence making the process cost effective and industrially viable.

### SUMMARY OF INVENTION

First aspect of the present invention provides a process for the preparation of Voclosporin of formula (I), comprising:
a) oxidation of compound of formula (III) with a mixture of potassium osmate and N-methyl orpholine N-oxide (NMMO) to form dihydroxy compound of formula (IVa);

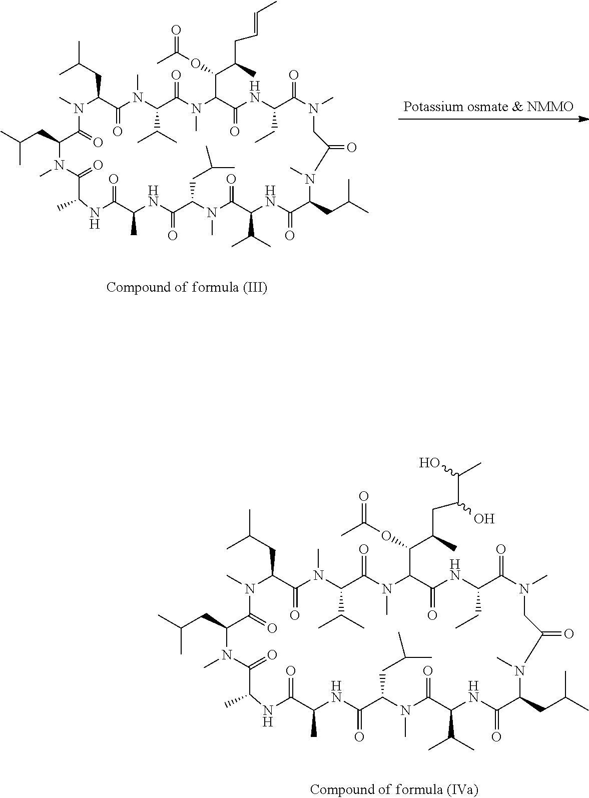

b) converting the dihydroxy compound of formula (IVa) to Voclosporin of formula (I).

Second aspect of the present application provides a process for the preparation of compound of formula (IVa), comprising oxidation of compound of formula (III) with a mixture of potassium osmate and N-methyl morpholine N-oxide (NMMO) to form dihydroxy compound of formula (IVa)

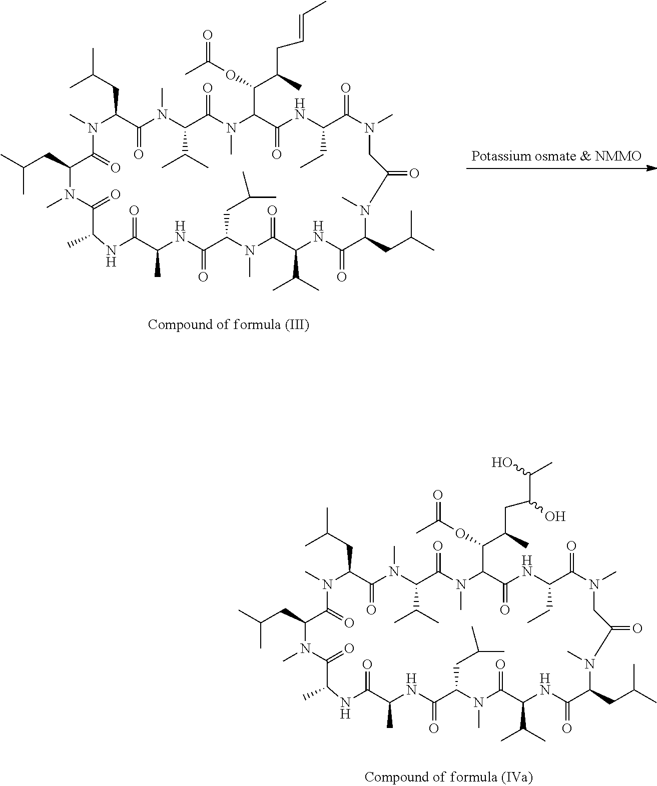

Third aspect of the present application provides a process for preparation of pure Voclosporin comprising crystallization of crude Voclosporin from a mixture of ketone solvent and hydrocarbon solvent.

### DETAILED DESCRIPTION OF INVENTION

First aspect of the present invention provides a process for the preparation of Voclosporin of formula (I), comprising:
a) oxidation of compound of formula (III) with a mixture of potassium osmate and N-methylmorpholine N-oxide (NMMO) to form dihydroxy compound of formula (IVa);

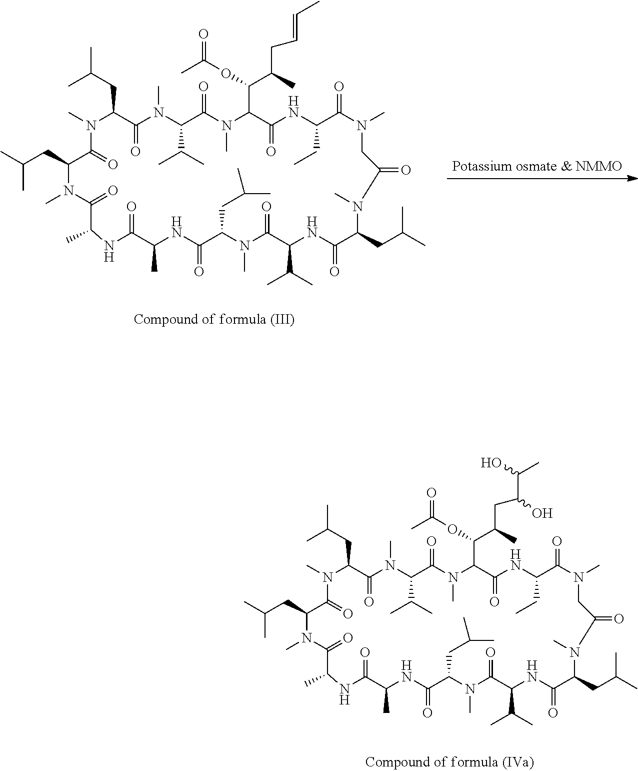

b) converting the dihydroxy compound of formula (IVa) to Voclosporin of formula (I).

In one embodiment, the oxidation reaction may be carried out by a process comprising stirring the reaction mixture of compound of formula (III), potassium osmate, N-methylmorpholine N-oxide (NMMO) in a solvent. The reaction mixture containing potassium osmate and aqueous N-methyl morpholine N-oxide solution (NMMO) in-situ generates osmium tetroxide (OsO₄) which enables oxidation of compound of formula (III) to form dihydroxy intermediate of formula (IVa). The step may be carried out in a solvent which includes but not limited to an ether solvent such as methyl tert-butyl ether, diethyl ether thereof; an alcohol solvent such as methanol, ethanol, isopropanol, n-butanol, t-butanol thereof; an ester solvent such as methyl acetate, ethyl acetate thereof; and the like or mixture thereof. Specifically, the solvent is a mixture of methyl tert-butyl ether and t-butanol. The reaction may be carried out for about 1 hour to about 24 hours at a temperature of about 15° C. to about boiling point of the solvent used to obtain the compound of formula (IVa). Specifically, the reaction mixture may be stirred for about 12 hours at about 25° C.

In other embodiment, N-methylmorpholine N-oxide (NMMO) may be replaced with other similar astoichiometric re-oxidants selected from the group comprising organic and inorganic peroxides such as hydrogen peroxide and tert-butyl hydroperoxide and the like; inorganic chlorates such as sodium chlorate, potassium chlorate and the like; air and oxygen and the like; potassium ferricyanide and the like; peracids and the like.

In another embodiment, the compound of formula (IVa) may be isolated and optionally crystallized before it is further oxidized to form compound of formula (IV) in presence of sodium periodate.

In another embodiment, in-situ oxidation of dihydroxy compound of formula (IVa) may be carried out with sodium periodate to provide compound of formula (IV)

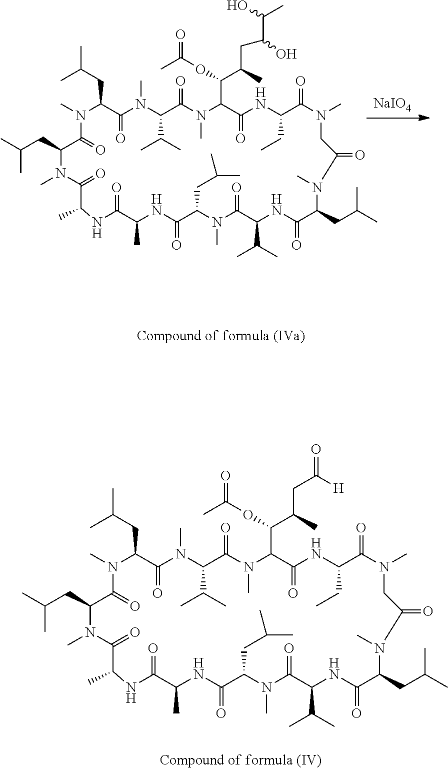

In another embodiment, the oxidation reaction of compound of formula (IVa) may be carried out by a process comprising stirring a reaction mixture of compound of formula (IVa) and sodium periodate for about 1 hour to about 24 hours at a temperature of about 15° C. to about boiling point of the solvent to obtain the compound of formula (IV). Specifically, the reaction mixture may be stirred for about 12 hours at about 25° C. The oxidation reaction of compound of formula (IVa) with sodium periodate may be carried out in a suitable solvent. Specifically, the oxidation reaction of compound of formula (IVa) may be carried out in the same solvent in which oxidation of compound of formula (III) has been taken place.

In yet another embodiment, conversion of compounds of formulae (IVa) and (IV) to Voclosporin of formula (I) may be carried out by the methods known from the examples of present application and also prior art patents such as U.S. Pat. Nos. 7,332,472 B2 and 7,799,756 B2.

Second aspect of the present application provides a process for the preparation of compound of formula (IV), comprising; oxidizing of compound of formula (III) using potassium osmate, N-methylmorpholine N-oxide (NMMO) in a solvent to produce dihydroxy compound of formula (IVa) which further treated with sodium periodate to form compound of formula (IV).

In another embodiment, the compound of formula (IV) is further converted to Voclosporin of formula (I), comprising:
a) reacting the compound of formula (IV) with tributyl phosphonium allyl bromide in presence of potassium tert-butoxide & tetrahydrofuran to provide the compound of formula (V);

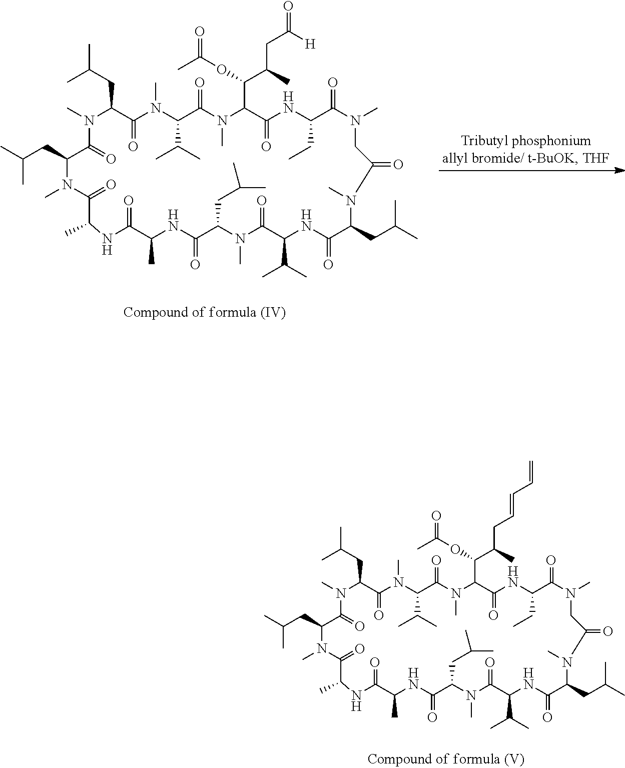

b) treating the compound of formula (V) with potassium carbonate in a mixture of methanol & water to provide the compound of formula (I)

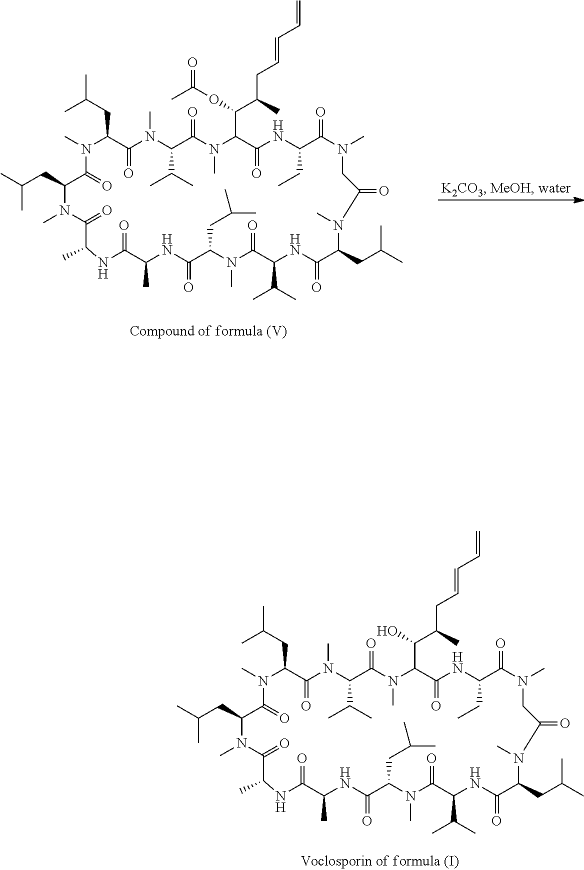

Third aspect of the present application provides a process for preparation of pure Voclosporin comprising crystallization of crude Voclosporin from a mixture of ketone solvent and hydrocarbon solvent.

The ketone solvent is selected from a group of acetone, butanone, methyl ethyl ketone and methyl isobutyl ketone; the hydrocarbon solvent is selected from a group of n-heptane, n-hexane, toluene and the like.

The present application provides a process for preparation of pure Voclosporin comprising crystallization of crude Voclosporin using a ketone solvent as a solvent and a hydrocarbon solvent as an anti-solvent.

In one embodiment, wherein the ketone solvent is methyl ethyl ketone.

In another embodiment, wherein the hydrocarbon solvent is n-heptane.

The crystallization/isolation of Voclosporin may be performed using different techniques known in the art e.g. evaporation, concentration, filtration of isolated solid and the like. Suitable temperatures for isolation may be about 20° C. or any other suitable temperatures. Filtration can be achieved by any means known in the art. The obtained solid may optionally be dried. The drying may be carried out at atmospheric pressure or above, or under reduced pressures, specifically at temperatures less than about 80° C. and more specifically less than about 60° C. and most specifically at about 40° C. The drying may be carried out for any time period required for obtaining a desired product quality, such as from about 5 minutes to about 24 hours, or longer.

### DEFINITIONS

The following definitions are used in connection with the present invention unless the context indicates otherwise.

The term “about” when used in the present application preceding a number and referring to it, is meant to designate any value which lies within the range of ±10%, preferably within a range of ±5%, more preferably within a range of ±2%, still more preferably within a range of ±1% of its value. For example, “about 10” should be construed as meaning within the range of 9 to 11, preferably within the range of 9.5 to 10.5, more preferably within the range of 9.8 to 10.2, and still more preferably within the range of 9.9 to 10.1 at they modify as those terms are understood by those of skill in the art. This includes, at very least, the degree of expected experimental error, technique error and instrument error for a given technique used to measure a value.

All percentages and ratios used herein are by weight of the total composition and all measurements made are at about 25° C. and about atmospheric pressure, unless otherwise designated. All temperatures are in degrees Celsius unless specified otherwise. As used herein, the terms “comprising” and “comprises” mean the elements recited, or their equivalents in structure or function, plus any other element or elements which are not recited. The terms “having” and “including” are also to be construed as open ended. All ranges recited herein include the endpoints, including those that recite a range between two values. Whether so indicated or not, all values recited herein are approximate as defined by the circumstances, including the degree of expected experimental error, technique error, and instrument error for a given technique used to measure a value.

Schematic representation of Voclosporin synthesis of the present invention is shown below.

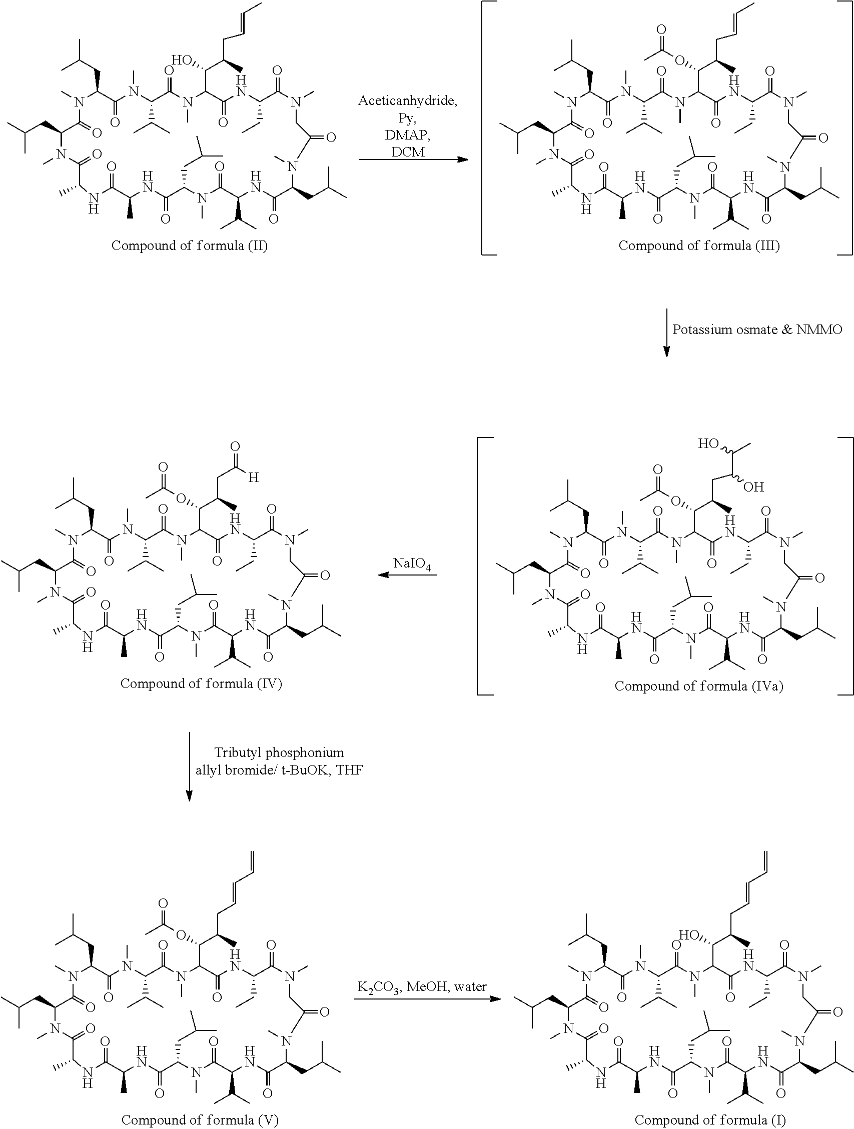

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present invention. While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES

### Example 1: Preparation of Cyclosporin A Acetate of Formula (III)

A mixture of Cyclosporine A (10.4 g), 4-dimethylaminopyridine (1.056 g), dichloromethane (42 ml), pyridine (6.84 g) and acetic anhydride (8.83 g) were stirred for about 16 hrs at 20-25° C. Quenched the reaction mixture with aqueous sodium bicarbonate solution. Separated the organic and aqueous layers. Extracted the aqueous layer with methyl tert-butyl ether. Combined the total organic layers and washed sequentially with aqueous hydrochloride solution (1M), aqueous sodium bicarbonate solution, water followed by aqueous brine solution. Separated the organic layer and dried over sodium sulfate. Distilled off the solvent completely from organic layer under reduced pressure to afford title compound as a white solid (Yield: 10.36 g).

### Example 2: Preparation of Cyclosporin A Acetate Aldehyde Compound of Formula (IV)

A mixture of cyclosporin A acetate (10.3 g), methyl tert-butyl ether (17 mL), t-butanol (3.6 mL), aqueous N-methyl morpholine N-oxide solution (1.415 g of a 50% solution in 2.52 mL of water) and potassium osmate dihydrate (0.076 g) were stirred for about 16 hours at 20-25° C. Quenched the reaction mixture with aqueous sodium thiosulfate solution. Separated the organic and aqueous layers. Extracted the aqueous layer with methyl tert-butyl ether. Combined the total organic layers and washed with brine solution. Separated the organic layer (Contains dihydroxy compound of formula (IVa); exact mass: 1277.8 daltons) and diluted by addition of water (60 mL) and acetic acid (0.4 mL).

Sodium periodate (2.43 g) was added to the reaction mixture and stirred for about 18 hours at 20-25° C. Washed the reaction mixture with brine solution (20 mL) followed by water (20 mL). Separated the organic and aqueous layers. Extracted the aqueous layers with methyl tert-butyl ether (60 mL). Combined the total organic layers and dried over sodium sulfate. Distilled off the organic layer under reduced pressure to afford compound of formula (IV) (Yield: 10.07 g, 98.7%).

The obtained crude compound of formula (IV) was subjected to column chromatography (Combiflash, heptane/acetone gradient) to afford a white solid (Yield: 8.98 g, 88%).

A fraction of crude compound of formula (IV) (3.2 g) (another batch, which is prepared by the same process) was purified by dissolved in methanol (10 mL) at 20-25° C. and then slowly added to water (115 mL). Filtered the precipitated solid and washed with water to afford pure solid compound (Yield: 2.77 g).

### Example-3: Preparation of Acetyl Voclosporin of Formula (V)

Cyclosporin A acetate aldehyde compound of formula (IV) (5.14 g) was dissolved in anhydrous THE solution (31 mL) at 20-25° C. and added to tributyl phosphonium allyl bromide (3.48 g). Cooled the reaction mixture to −65° C. and added potassium tert-butoxide (9.7 mL, 1 M in THF) dropwise over 9 minutes. The reaction mixture was stirred for about 3 hours at −65° C. Toluene (25 ml) and saturated ammonium chloride solution (25 mL) were added and stirred for about 10 min at −50° C. Transferred the reaction mixture to a separating funnel and washed the flask across with toluene. Separated the organic and aqueous layers and extracted the aqueous layer with toluene. Combined the total organic layers and washed with aqueous sodium chloride solution (20 mL). Separated the organic layer and dried over sodium sulfate. Distilled off the organic layer under reduced pressure (40-45° C., 2 mbar) and co-distilled with methanol (50 mL) to afford white solid compound (5.67 g).

### Purification

The above obtained crude compound was dissolved in methanol (33.37 mL) and added water (17.7 mL) to it followed by stirred for about 2 hours at 20-25° C. Filtered the precipitated solid and washed with mixture of methanol & water. Washed the obtained solid with dichloromethane (3*20 mL). Separated the organic and aqueous layers. Extracted the aqueous layer with dichloromethane. Combined the total organic layers and dried over sodium sulfate and distilled off the solvent under reduced pressure. Co-distilled with dichloromethane/methyl tert-butyl ether (1:1 v/v; 50 mL). Further, co-distilled with methyl tert-butyl ether (50 mL) to afford white solid compound (4.09 g).

### Example 4: Preparation of Voclosporin

Acetyl Voclosporin (2.876 g) was dissolved in methanol (67.3 mL) at 20-25° C. Water (27 mL) and potassium carbonate (2.71 g) were added to the obtained mixture and stirred for about 60 hours at 20-25° C. Distilled off the reaction mixture partially and added ethyl acetate (50 ml) and aqueous ammonium chloride solution (25 ml). Separated the organic and aqueous layers and extracted the aqueous layer with ethyl acetate. Combined the total organic layers and dried over magnesium sulfate. Distilled off the organic layers under reduced pressure. The obtained Voclosporin residue compound was dissolved in a mixture of acetone (6 mL) and n-heptane (9 mL) followed by evaporation of the solvent under reduced pressure to afford Voclosporin as a white solid (2.27 g).

### Example-5: Preparation of Cyclosporin A Acetate Aldehyde Compound of Formula (IV)

A mixture of Cyclosporine A (50 g), 4-dimethylaminopyridine (5 g), dichloromethane (200 ml), pyridine (23.4 ml) and acetic anhydride (27.4 g) were stirred for about 24 hrs at 25-30° C. Quenched the reaction mixture with 10% aqueous sodium bicarbonate solution. The organic and aqueous layers were separated and the aqueous layer was extracted with methyl tert-butyl ether. The total organic layers were combined and sequentially washed with 10% aqueous hydrochloride solution, 10% aqueous sodium bicarbonate solution, water followed by 10% aqueous brine solution respectively. The organic layer was separated and dried over sodium sulfate. Distilled off the solvent from organic layer under reduced pressure to afford cyclosporin A acetate of formula (III).

Methyl tert-butyl ether (200 mL), t-butanol (17.5 mL), aqueous N-methyl morpholine N-oxide 50% solution (19.49 ml), potassium osmate dihydrate (0.38 gms) and water (11 ml) were added to the above obtained cyclosporin A acetate compound and stirred for about 22 hours at 25-30° C. The reaction mixture was quenched with aqueous sodium thiosulfate solution. The organic and aqueous layers were separated and extracted the aqueous layer with methyl tert-butyl ether. The total organic layers were combined and washed with 10% brine solution and water simultaneously. The organic layer (contains dihydroxy compound of formula (IVa); exact mass: 1277.8 daltons) was diluted with water (250 ml) and acidified with acetic acid (2.5 ml).

Sodium periodate (14.23 gms) was added to the above obtained reaction mixture and stirred for about 22 hours at 25-30° C. The organic layer was separated and washed with 10% brine solution followed by water. Partially distilled off the solvent from organic layer and slurried in n-heptane (500 ml) for about 90 min at 25-30° C. The solid obtained was filtered and washed with n-heptane and then dried to afford title compound (Yield: 49gms, 95%).

### Example-6: Preparation of Acetyl Voclosporin of Formula (V)

Cyclosporin A acetate aldehyde compound of formula (IV) (50.0 gms) was dissolved in anhydrous THF solution (100 mL) at 25-30° C. and added to tributyl phosphonium allyl bromide (33.83 gms). Anhydrous THE solution (100 mL) was added to the above reaction mixture and cooled to −65° C. and then added potassium tert-butoxide (94.51 mL). The reaction mixture was stirred for about 6 hours at −65° C. Toluene (250 ml) was added at below −30° C. followed by addition of 20% saturated ammonium chloride solution (250 mL) and increased the temperature to 0-5° C. and stirred for 30 min. The reaction mixture was transferred to a separating funnel and washed the flask across with toluene. The organic and aqueous layers were separated and extracted the aqueous layer with toluene. The total organic layers were combined and washed with 10% aqueous sodium chloride solution. Distilled off the solvent from organic layer under reduced pressure and co-distilled with methanol (1000 mL).

### Purification

The above obtained crude compound was dissolved in methanol (330 mL) and added water (172.5 mL) followed by stirred for about 3 hours at 25-30° C. The precipitated solid was filtered and washed with mixture of methanol & water. Methyl tert-butyl ether (500 ml) was added to the above obtained solid compound at 25-30° C. and heated to 40-45° C. and stirred for about 90 min. at same temperature. The reaction mixture was allowed to 25-30° C. and stirred for about 3 hours. The solid obtained was filtered, washed and then dried to get acetyl Voclosporin of formula (V) (Yield: 37 gms, 73%)

### Example 7: Preparation of Voclosporin

Acetyl Voclosporin (50 gms) was dissolved in methanol (1150 mL) at 25-35° C. Aqueous potassium carbonate solution (47.2 gms dissolved in 500 ml of water) was added to the obtained mixture and stirred for about 24 hours at 25-35° C. Distilled off the reaction mixture partially and added ethyl acetate (500 ml) and 20% aqueous ammonium chloride solution. The organic and aqueous layers were separated and the aqueous layer was extracted with ethyl acetate. The total organic layers were combined and washed with 10% brine solution. Distilled off the solvent completely from organic layers under reduced pressure and co-distilled with acetone (2*250 ml). The obtained Voclosporin residue compound was dissolved in acetone (100 mL) and slowly n-heptane (425 mL) was added and then stirred for about 6 hours. The precipitated solid was filtered, washed with n-heptane and then dried to afford Voclosporin. The obtained Voclosporin was dissolved in acetone (70 ml) and slowly added n-heptane (297.5 ml). The obtained mixture was stirred for about 6 hours at 25-30°° C. The precipitated solid was filtered, washed with n-heptane and the dried to afford pure Voclosporin (Yield: 30 gms, 62%, and E/Z ratio: 92:8)

We claim:

1. A process for the preparation of Voclosporin of formula (I), 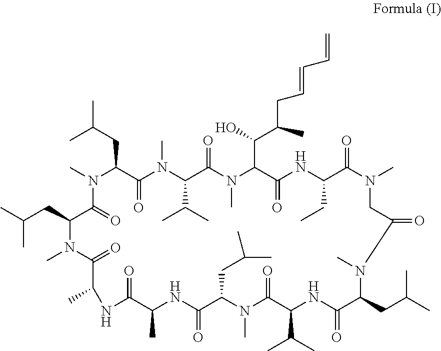 [SMILES: C=C/C=C/C[C@@H](C)[C@@H](O)C1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
comprising:
a) oxidizing compound of formula (III) with potassium osmate and N-methylmorpholine N-oxide (NMMO) to form dihydroxy compound of formula (IVa); 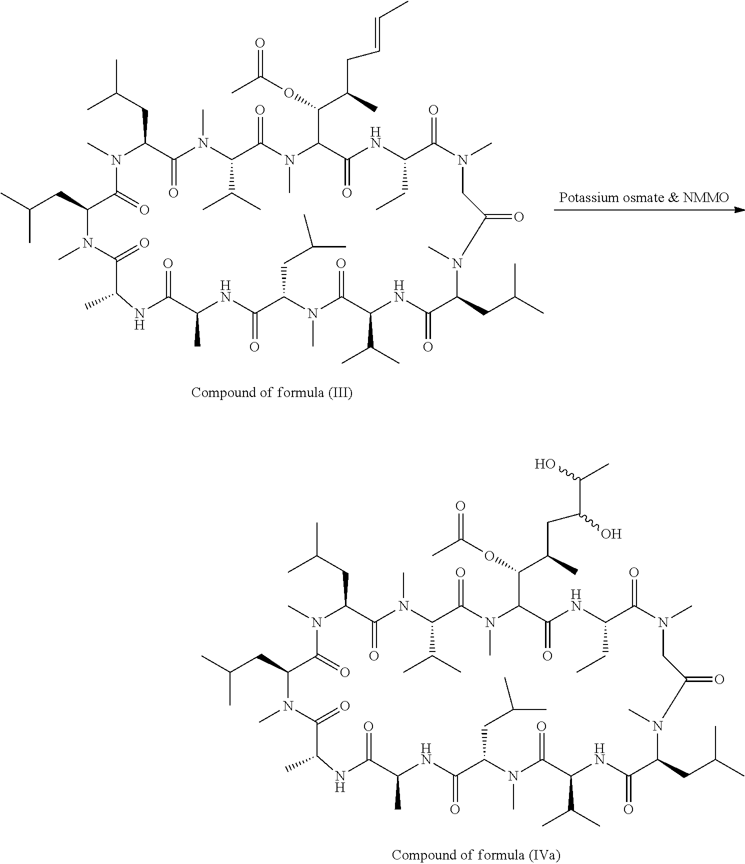
b) converting the dihydroxy compound of formula (IVa) to Voclosporin of formula (I).

2. The process of claim 1, wherein step-a) reaction is carried out in a solvent mixture comprising methyl tert-butyl ether and t-butanol.

3. The process of claim 1, wherein the compound of formula (IVa) is further oxidized to form compound of formula (IV) in presence of sodium periodate.

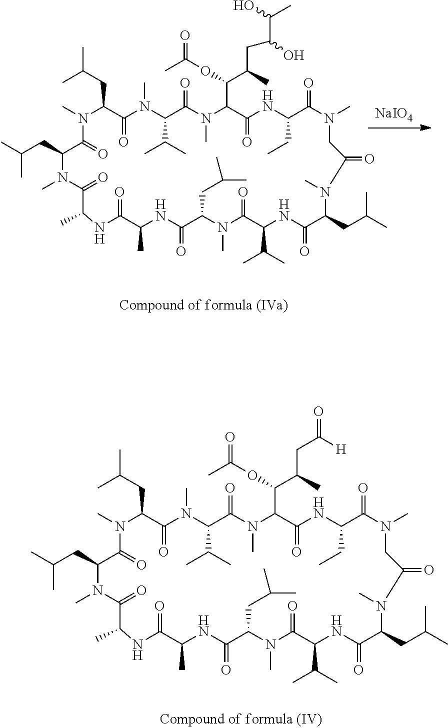

4. The process of claim 1, further comprises conversion of compound of formula (IVa) to Voclosporin of formula (I), comprising:
a) oxidizing the compound of formula (IVa) with sodium periodate (NaIO₄) to form the compound of formula (IV); 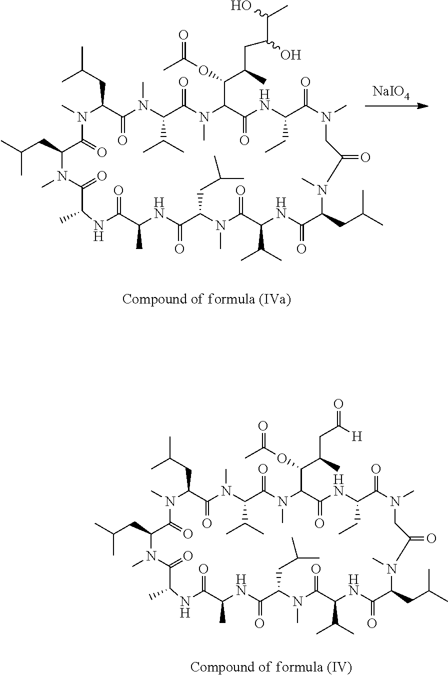
b) reacting the compound of formula (IV) with tributyl phosphonium allyl bromide in presence of potassium tert-butoxide & tetrahydrofuran to provide the compound of formula (V); 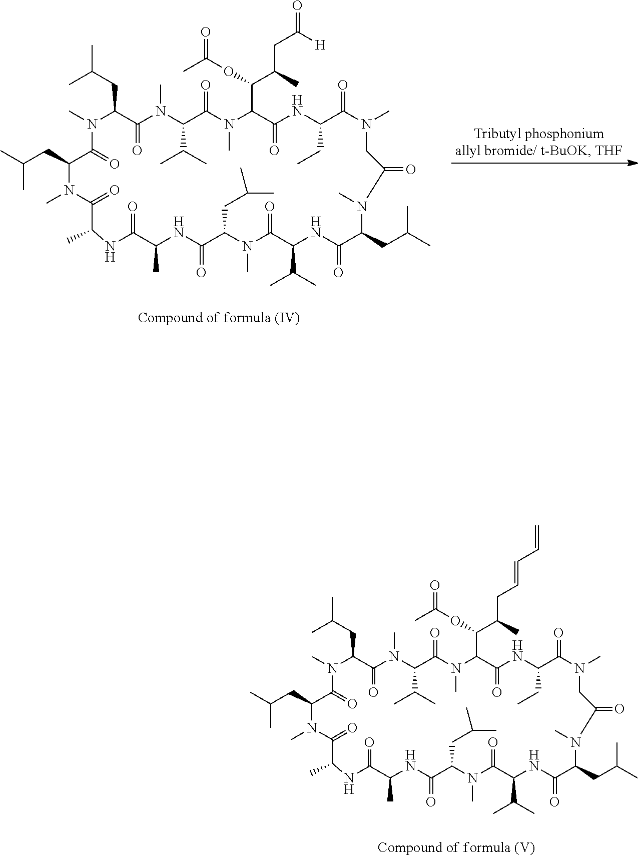
c) treating the compound of formula (V) with potassium carbonate in a mixture of methanol & water to provide the compound of formula (I).

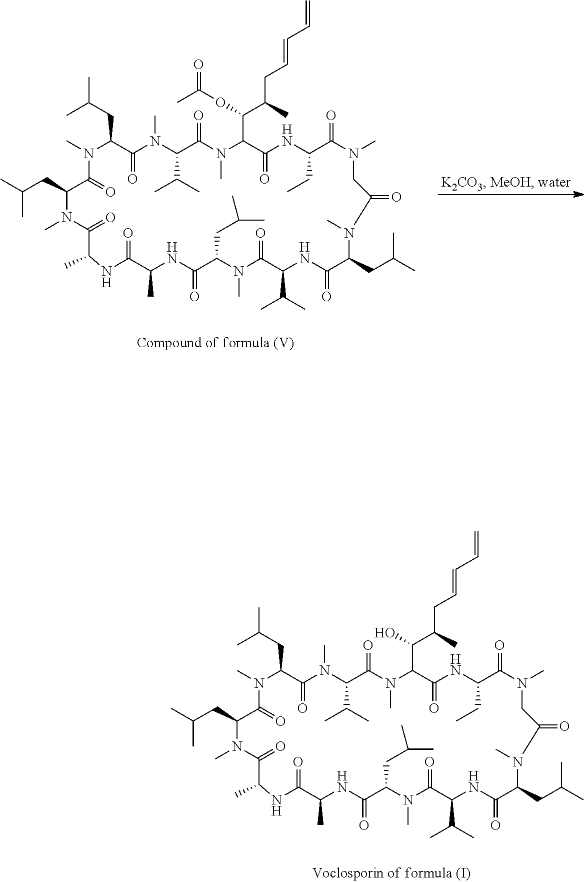

5. A process for the preparation of compound of formula (IV), 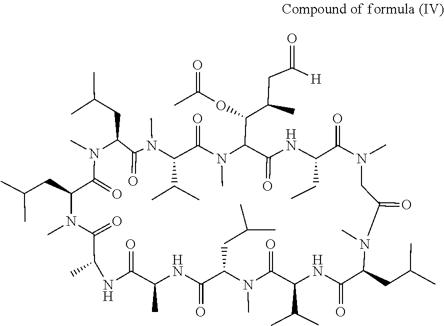 [SMILES: CC[C@@H]1NC(=O)C([C@H](OC(C)=O)[C@H](C)CC=O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]
comprising:
a) oxidizing compound of formula (III) with potassium osmate and N-methylmorpholine N-oxide (NMO) in a solvent to produce dihydroxy compound of formula (IVa) 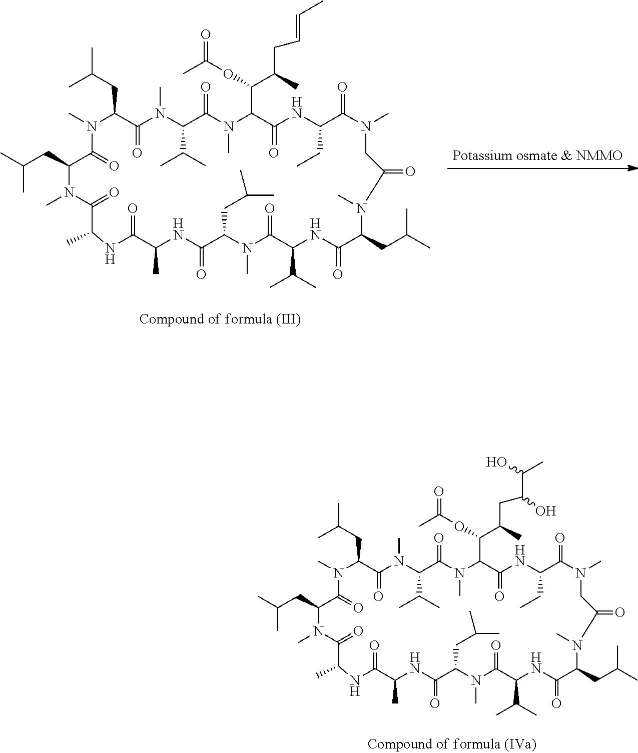
b) treating the compound of formula (IVa) with sodium periodate to get compound of formula (IV)

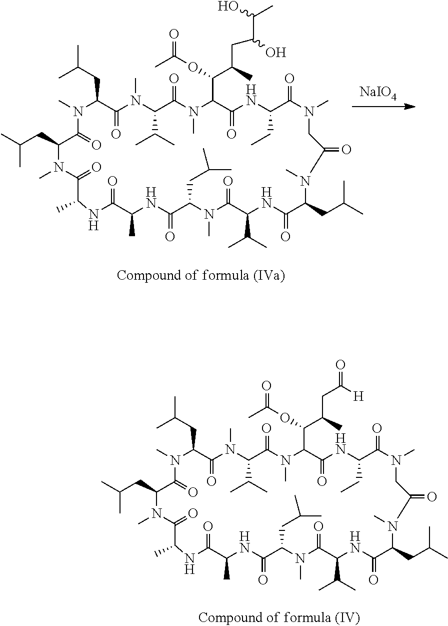

6. The process of claim 5, wherein step-a) reaction is carried out in a solvent mixture comprising methyl tert-butyl ether and t-butanol.
